(19) United States
(12) Patent Application Publication
(10) Pub. No.: US 2019/0252062 A1
(43) Pub. Date: Aug. 15, 2019

(54) Patient Communications System and Method

(71) Applicant: Geri Brumit, Fort Collins, CO (US)

(72) Inventor: Geri Brumit, Fort Collins, CO (US)

(21) Appl. No.: 15/894,704

(22) Filed: Feb. 12, 2018

(51) Int. Cl. G16H 40/20; G16H 10/60; A61B 5/00

(52) U.S. Cl. CPC G16H 40/20; G16H 10/60; G16H 40/60; A61B 5/7465; A61B 5/7445

(57) ABSTRACT

Disclosed is a method is a system for use of a display of medical information in a medical room. A computer application shows emergent medications and electrical shock dosages for coding patients based on a patient's size and age. Information is transferrable to a display mechanism viewable by multiple medical staff at one time. The display mechanism may accept input from the medical staff and to send and receive information from the computer application. The display of information on the display mechanism may be altered in response to input from medical staff.

### FIELD OF THE INVENTION

The present invention relates generally to emergency room communications related to codes and critical care for patients.

### BACKGROUND OF THE INVENTION

Hospital emergency personnel often are involved in situations dealing with highly sensitive patients who may require care for conditions that are considered critical. Hospital emergency personnel may be involved in multiple codes involving infants, children, adults and the elderly. A code is generally when an individual does not have a pulse and may not he breathing. When a patient stops breathing, that patient may be transported to an emergency setting, such as a trauma room, where the hospital emergency personnel may have access to equipment that may be best suited to provide emergency care to the patient,

A situation involving a patient that is coding is stressful and chaotic. However, trained personnel, including doctors and nurses can deliver some organization to the chaos. A doctor can provide direction to emergency staff, such as orders for a particular medication for a particular patient, which the emergency staff can follow to help a patient survive a coding situation.

In some situations, a patient may arrive at an emergency center giving the emergency staff no warning of their arrival. The patient may be an unresponsive child or adult. Generally, such a patient will be moved to a trauma room. In such situation, for example, weight-based medications may be required.

### SUMMARY OF THE INVENTION

An embodiment of the invention may therefore comprise a system for providing care critical information to emergency medical personnel, the system comprising a processor capable of accessing an application, the application being capable of processing information related to a patient, and one or more display mechanisms situated in a room, the display being capable of receiving processed information from the processor and of receiving interactive input from a medical staff member, wherein interactive input from received at the display mechanism is relayed to the processor and the processor is enabled to alter the display format of the information at the display mechanisms.

An embodiment of the invention may further comprise a method for providing care critical information to emergency medical personnel, the system comprising inputting information regarding a coding patient into a program, outputting, via the program, processed information to a display mechanism, the processed information comprising a recommended dosage of at least one of a medication and an electrical input to be administered to the patient, accepting tactile input from the medical personnel at the display mechanism, and updating the display mechanism based on the tactile input via the program.

An embodiment of the invention may further comprise a computer implemented method for providing critical care information to medical personnel, the method comprising in a computer system, receiving prestored information at a presentation point, the prestored information comprising a database of protocols, inputting specification data into the system, the specification data comprising data related to an individualized incident, correlating the prestored information based on the input specification data, wherein the step of correlating the prestored information based on the input specification data comprises eliminating first portions of the prestored information from an updated protocol and selecting second portions for use in the updated protocol, inputting event information into the system, wherein the input information causes the system to re-correlate the pre-stored information and calculate an updated correlated data specifically based on the specification data and the event data, presenting the updated correlated data at the presentation point.

### BRIEF DESCRIPTION OF THE DRAWINGS

FIG. 1 shows a flow diagram of an embodiment of the invention.

FIG. 2 shows a system for providing care critical information to emergency medical personnel.

FIG. 3 shows an embodiment of a system for providing care critical information to emergency personnel.

### DETAILED DESCRIPTION OF THE EMBODIMENTS

In emergency situations, correct medications and medical care is required for patients that need emergency care. Often, medical procedures may be instituted in such situations before complete information on the patient is developed. Eliminating as much human error in the delivery of medical care is required.

In an emergency situation, a documenting nurse may enter information related to the care and condition of a number of patients that have entered an emergency facility and trauma rooms. The documenting nurse has access to a computer screen that is not accessible to other emergency medical personnel that are moving about performing lifesaving care.

In an embodiment of the invention, a touch screen is enabled, through a software program accessible via “the cloud,” to compile patient information and access instant medication information generalized for that patient. For ease of use, this description may refer to such a computer program as Dose Perfect. For instance, a nurse may enter the weight of a patient into Dose Perfect. If the weight of the patient is not known, a nurse may use a Broselow tape (for pediatrics) or a previous weight to estimate for elderly or adult patients. Upon entry of the patient's weight, all emergent medications will pop up on the interactive screen of the Dose Perfect. The Dose Perfect board will be a board capable of displaying the information from the Dose Perfect program in a location, and at a size, that is viewable to the entire medical staff in the trauma room. For instance, a Dose Perfect screen may be on a wall where it is visible to the entire medical staff needing current updated information.

In an example of a coding or emergent patient, while medical staff is intubating a patient that has ceased breathing, the medical staff can refer to a visible screen to see suitable medications and doses while providing direction to other staff members in the trauma room. A Dose Perfect program and board eliminates the need for transfer of information in a give and take type manner from the documenting nurse to the emergency medical provider. In a situation where a doctor desires a coding patient be given a certain medication, the doctor can verbally order the medication and dosage after referring to the Dose Perfect board without having to make dosage conversions in his head during an emergent situation. Accordingly, human error may be mitigated. The appropriate medications and dosages are already on the Dose Perfect screen for the coding patient and the conversion to the dosage and time range that a nurse needs to administer the medication is provided on the Dose Perfect board by the Dose Perfect program.

In an embodiment of the invention, the converted medication and dosage is presented on the Dose Perfect board. Upon administration of the prescribed medication by an emergency staffer, that staffer can interact with the Dose Perfect screen. The staffer can touch the medication on the Dose Perfect board when the medication has been delivered. The interaction with the Dose Perfect board will provide an indication on the Dose Perfect hoard that the medication has been given and may indicate a time that the medication was given. For instance, the Dose Perfect board may indicate administration of the medication by highlighting the medication and providing a date stamp next to the medication. Next dosage timing information may also be indicated on the Dose Perfect board via the Dose Perfect screen.

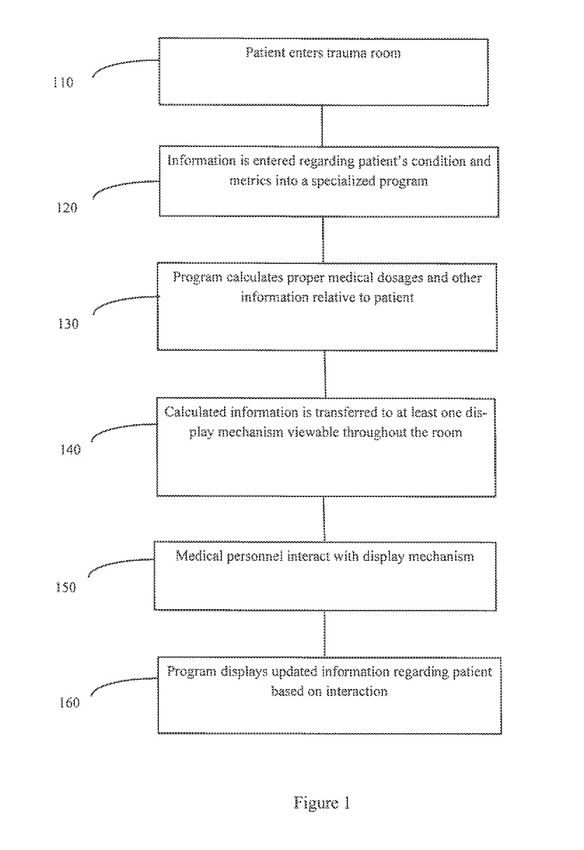

FIG. 1 shows a flow diagram of an embodiment of the invention. At step 110, a patient comes into an emergency center and is taken to a trauma room. A documenting staff member will enter information into a computer at step 120. The computer is enabled to operate a program that provides calculations related to medical dosages, both pharmaceutical and electrical, for the patient based on the information entered into the program. For instance, the information may include the size and weight, and approximate age of a patient. It is understood that any information that may be relevant to a medical dosage may be entered into the program and the program may be capable of utilizing a variety of information related to patient specifics and dosages. At step 130, the program calculates the proper medical dosages and other information relevant to the patient. This relevant information may include timing for multiple dosages as well as a time constraint for any particular dosage. Those skilled in the art of emergency medical care will understand the variables relative to proper medical dosages and administration. At step 140, the calculated information from the program is transferred to a display mechanism viewable throughout the room. The display mechanism may be wirelessly connected to the computer that hosts the program. The display mechanism may also be connected in a wired manner to the computer that hosts the program. The display mechanism may be any type of display mechanism that allows user interaction, such as a touch screen or a smart board. At step 150, medical personnel interact with the display mechanism. The medical personnel interaction may be to touch the display mechanism to indicate an action has occurred. The interaction may send a response to the program which may calculate new information for display. Such new information may include, timing information for a next dosage. The display mechanism may also indicate that an action has taken place to notify all medical personnel. Accordingly, human error in administration of medications may be reduced. At step 160, the program will display updated information regarding the patient based on the interaction from the medical personnel. As noted, the updated information may relate to timing information for next dosages. Further, the updated information may provide color coding of information on the display mechanism. For instance, when medical personnel interact with the display mechanism by touching a particular piece of information, that piece of information may change colors to indicate that a dosage has been given. Also, information that has not yet been acted upon may be in a different color. And further, information related to future actions may be in yet a third color. It is also understood that display indications may be in any format that provides information to medical personnel. For instance, other than color, font or text size may be used to indicate needed, or taken, actions. The program may be customizable by a user, or staff, to provide indications in any manner which best suits a particular medical staff or facility.

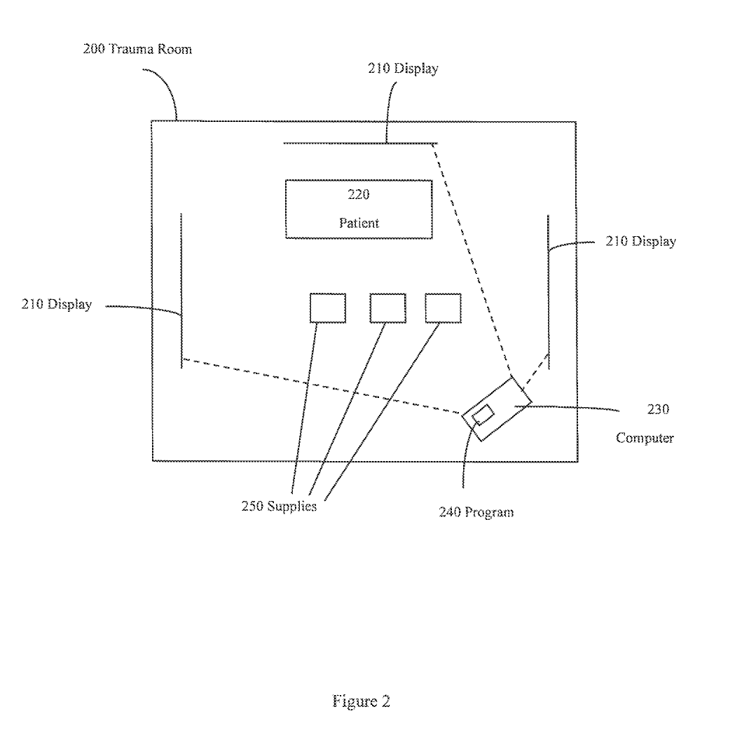

FIG. 2 shows a system for providing care critical information to emergency medical personnel. A trauma room 200 may have a plurality of display mechanisms 210 situated around the room 200. The room 200 will have a patient location 220. The display mechanisms 210 may he connected to a computer 230 which hosts a program 240. The trauma room may have a supply of medical equipment and medications 250. The computer 230 may be a general-purpose computer or handheld device capable of easily and efficiently inputting information relevant to a patient on the table 220. The computer may communicate with each of the displays in a wireless manner or in a wired manner. The displays 210 may be arranged around the room 200 in any manner that is suitable to visualization by medical personnel in the room 200. There are three displays 210 shown in FIG. 1. However, it is understood that there may be more or less displays 210 depending on the preferences of the medical staff and administration and the layout of a particular room 200. Information from the program 240 is relayed to the displays 210 where medical staff (not shown) can interact with the displays 210. Information from interaction by medical staff with the displays 210 will be relayed back to the program where the displayed information can be updated. Color coding, or another method of distinguishing messages visually, may be used in the Dose Perfect board to provide easily translatable information to emergency staff personnel.

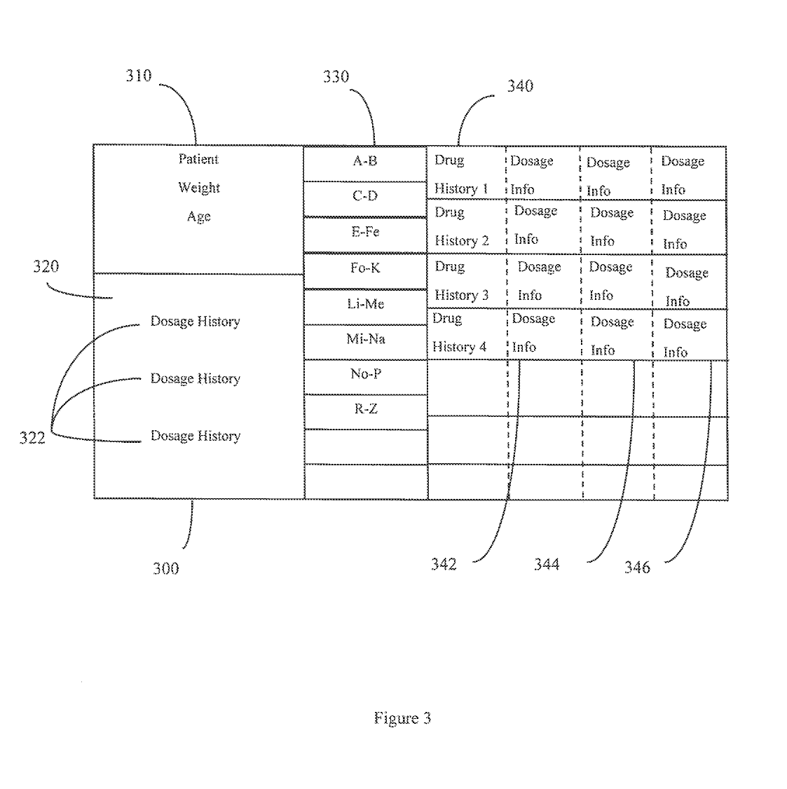

FIG. 3 shows an embodiment of a system for providing care critical information to emergency personnel. A system of an embodiment of the invention may comprise a display 300. The display 300 may be divided up into different sections that provide different information to emergency personnel. A patient identification 310 may provide information regarding the patient's weight and age. A dosage history 320 may provide information regarding the history of dosages previously given to the patient. An emergency personnel enters a dosage of a medication into the system. Each dosage entry 322 may be timestamped to provide information regarding the timing of dosage events. Each entry 322 may be independent of other entries 322 in the dosage history 320 section. As such, a first entry may indicate that a certain dosage of Drug1 was provided to a patient at a first time. A second entry may indicate that a certain dosage of Drug2 was provided to a patient at a second time. It is understood that the first and second time may be the same time if the Drug1 and Drug2 were administered at the same time.

The embodiment in FIG. 3 may also comprise a easy reference 330 for a listing of available medications. The easy reference 330 for the listing of available medications may be alphabetized. As shown in FIG. 3, the easy reference 330 is shown as an alphabetic guide. For instance, medications may fall under one of the easy references in the alphabetic spectrum. Hitting the C-D easy reference may provide a listing of available medications that begin with C or D. The break-up of the alphabet may be by any delineation that a user desires.

Continuing with FIG. 3, depending on a user's selections, a categorized drug dosage 340 for a range of drugs may be presented. It is understood that the categorization may be by an alphabetic selection, or by any other categorization that may be useful to a user. For the embodiment shown in FIG. 3, the categorization is alphabetic. For purposes of understanding the interactions of the different portions of the display 300, it is assumed that a user has selected one of the easy references 330 which are alphabetic. All the possible drugs, or medications, may be presented in the drug dosage portion 340 of the display. The drug dosage portion of the display may present information related to timing and dosages of medications progressively. For instance, a first dosage of a medication may indicate a first quantity and a second and third dosage may be indicated to only be given after a certain time has expired and only in limited dosages.

Embodiments of the invention provide a safety check on medications depending on the weight and age of a patient. As discussed, appropriate dosages are presented to emergency personnel. The appropriate dosages of different medications may comprise multiple administrations of a particular medication. Further, if information is known related to a particular patient's health status, that information may be entered into the system. For instance, if a patient is known to have an allergy to a particular medication, that information may be entered into the system to alert emergency medical personnel that an alternative medication may be preferred. As information is gathered, including administration of medication information, that gathered information may be input into the system and that information may be presented to users in a dedicated area of a presentation. That dedicated area may be a dosage history 320 area such as that shown in FIG. 3. It is understood that a dedicated area may be multiple areas of a presentation based on the type of information. For instance, allergy info nation may be presented in an area different than a dosage history information.

Color coding of the presented information may also be presented. For instance, non-approved patient medications may be presented in a color to indicate a certain status for the medication. That color may be red, for instance. The text may be presented in red or the background for the text may be presented in red. It is understood that other colors may be used. A color may also be used to indicate that an administration of a certain drug is permitted. This color may be green, for example. The text may be presented in green or the background for the text may be presented in green. For instance, in the dosage history 320 presentation, as shown in FIG. 3, color coding may be provided for different dosage histories to indicate whether administrations of different medications is acceptable. A Drug1 presentation may be provided in red, for example, to indicate that that particular Drug1 may not he provided to the patient at this time. However, a Drug2 may be presented in a different color to indicate that administration of Drug2 is acceptable. As the time increased from the last administration of Drug1, the color may change once a threshold is reached for acceptable re-administration of that Drug1.

In the instance that a particular medication is not suitable for a particular patient, whether because of allergy indications or otherwise, alternatives to that medication may be provided. An alternative medication may be provided in the drug dosage 340 presentation.

In an embodiment of the invention, medical equipment may also be presented to medical personnel. This medical equipment information may include intravenous sizing, tube size or other medical equipment that is suitable for patients based on age, weight, or other qualification.

In embodiments of the invention, each presentation is for an individual patient. As such, each patient presentation is individualized. Accordingly, at a medical station, a patient's information can be selected, and the medical personnel can concentrate on that particular patient. As a patient transitions to a different location, the previous locations may be utilized to present new information for a different patient. Further, as a patient is transitioned to a different location, current information may be transferred to that different location. For instance, a patient my enter an emergency room at a hospital and receive different medications that are entered into the system. The system presents the updated information regarding dosages and acceptable administrations. The patient may need to be transferred to a more specialized hospital, for example. Moreover, the transfer may entail transportation in an ambulance or in a helicopter. The current information may be provided to the transportation means and to the other hospital. Further, as the transportation means may administer additional treatments, the information regarding the additional treatments is also provided to the other locations that may need the information. The transmittal of information may be by any method that is useful. The secondary hospital may be enabled to download patient specific information based on any information that is relevant. For instance, the primary hospital may provide the transportation means and secondary hospital with a picture of the current status presentation. The primary hospital may provide the transportation means and secondary hospital with a code that enables the transportation means and secondary hospital to engage with the system. This may comprise the transportation means and secondary hospital connecting via a network to the system. This enables the transportation means and the secondary hospital to be prepared for arrival of the patient.

In embodiments of the invention, a presentation of the system may comprise any method of device that is capable of providing a visual representation to a medical staff. Examples of such methods and devices are visual display monitors, cell phone monitors, computer monitors, and other types of monitors known to those skilled in the art. It is understood that the monitors may be touch screen enabled to allow medical staff to enter data and manipulate elements of the display without access to a keyboard or other input device. Data input means for the system may also comprise voice activated input. Those skilled in the art of voice activated and voice recognition methods and systems will understand the implementation of such devices and system. A voice activated system may allow medical staff to input data into the system with limited tactile abilities at a particular time—for instance when all hands are needed during an emergency. Voice activation may be turned on and off either with a physical activator or via voice commands.

In a method of an embodiment of the system, in a computer system, prestored information may be received at a presentation point. The prestored information may comprise data and information related to medicines and medical equipment. The information may also include dosage and timing information. The dosage and timing information may be protocols related to proper administration of medicines and uses of medical equipment. Medical staff personnel may then input specification data into the system. The specification data may comprise data related to an individualized incident. For instance, the specification data may include medicines that a patient is allergic to or that may be inappropriate for a specific patient for other reasons. The prestored information is correlated according to the specification. Such correlation may comprise taking the prestored information and indicating that one or more medicines, or pieces of medical equipment, are unsuited to the particular patient. Medical staff will input event information into the system. The event information may comprise administration of a particular medicine or use of a particular piece of medical equipment. The system will update information to be presented based on the initial prestored information, the specification information and the event information. The updated information may comprise a dosage history and recommended further administrations depending on a patient's status.

The foregoing description of the invention has been presented for purposes of illustration and description. It is not intended to be exhaustive or to limit the invention to the precise form disclosed, and other modifications and variations may be possible considering the above teachings. The embodiment was chosen and described to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

What is claimed is:

1. A system for providing care critical information to emergency medical personnel, said system comprising:
a processor capable of accessing an application, said application being capable of processing information related to a patient; and
one or more display mechanisms situated in a room, said display being capable of receiving processed information from said processor and of receiving interactive input from a medical staff member;
wherein interactive input from received at said display mechanism is relayed to said processor and said processor is enabled to alter the display format of said information at said display mechanism.

2. The system of claim 1, wherein said display format of said information comprises at least one of color, size and font.

3. The system of claim 1, wherein said one or more display mechanisms receives processed information from said processor wirelessly.

4. The system of claim 1, wherein said processed information comprises at least one of medical dosages, medical dosage intervals and dosage rates.

5. The system of claim 1, wherein said interactive input comprises a touch of the screen from said medical staff individual.

6. A method for providing care critical information to emergency medical personnel, said system comprising:
inputting information regarding a coding patient into a program;
outputting, via said program, processed information to a display mechanism, said processed information comprising a recommended dosage of at least one of a medication and an electrical input to be administered to said patient;
accepting tactile input from said medical personnel at said display mechanism; and
updating said display mechanism based on said tactile input via said program.

7. The method of claim 6, wherein said method of updating said display mechanism comprises altering at least one of a color, size and font of at least one item of information displayed on said display mechanism.

8. The method of claim 6, wherein said method of outputting processed information to a display mechanism comprises outputting processed information to a display mechanism wirelessly.

9. The method of claim 6, wherein said processed information comprises at least one of a medical dosage, a medical dosage interval and a dosage rate.

10. A computer implemented method for providing critical care information to medical personnel, said method comprising:
in a computer system;
receiving prestored information at a presentation point, said prestored information comprising a database of protocols;
inputting specification data into said system, said specification data comprising data related to an individualized incident;
correlating said prestored information based on said input specification data, wherein said step of correlating said prestored information based on said input specification data comprises eliminating first portions of said prestored information from an updated protocol and selecting second portions for use in said updated protocol;
inputting event information into said system, wherein said input information causes said system to re-correlate the pre-stored information and calculate an updated correlated data specifically based on said specification data and said event data;
presenting said updated correlated data at said presentation point.

11. The method of claim 10, said method further comprising color coding said updated correlated data.

12. The method of claim 10, wherein said step of inputting event information comprises inputting event information by voice recognition.

13. The method of claim 10, said method further comprising forwarding said updated correlated data to a secondary presentation point wherein said secondary presentation point is enabled to access said updated correlated data and input new event information.

14. The method of claim 13, wherein said secondary presentation point is a mobile station.
